(19) United States
(12) Patent Application Publication
(10) Pub. No.: US 2020/0283733 A1
(43) Pub. Date: Sep. 10, 2020

(54) METHODS FOR PRODUCING CANCER STEM CELL SPHEROIDS

(71) Applicant: Stemtek Therapeutics SL, Derio (ES)

(72) Inventors: Angel Garcia Martin, Derio (ES); Olatz Leis Esnaola, Derio (ES); Juan Gumuzio Barrie, Derio (ES)

(73) Assignee: Stemtek Therapeutics SL, Derio (ES)

(21) Appl. No.: 16/306,153

(22) PCT Filed: Jun. 1, 2017
(86) PCT No.: PCT/EP2017/063399

§ 371 Date: Nov. 30, 2018

(30) Foreign Application Priority Data
Jun. 2, 2016 (GB) 1609663.8

(51) Int. Cl. C12N 5/095

(52) U.S. Cl. CPC C12N 5/0695; C12N 2501/11; C12N 2500/50; C12N 2513/00; C12N 2501/115

(57) ABSTRACT

The invention provides a method for producing a population of ready-to-use spheroid forming cancer cells, comprising: (i) growing cancer cells in suspension culture in a first culture medium on one or more first low-adhesion tissue culture plates thereby forming cancer cell spheroids enriched in cancer stem cells; (ii) disaggregating said cancer cell spheroids to form a suspension of single cells enriched in cancer stem cells; (iii) plating said suspension of single cells in a second culture medium on one or more second low-adhesion tissue culture plates; and (iv) freezing said suspension of single cells in said one or more second tissue culture plates, thereby producing a population of ready-to-use spheroid forming cancer cells. Also provided are cell populations produced by the method and kits for growing cancer cell spheroids, including for use in screening of test compound.

### FIELD OF THE INVENTION

The present invention relates to methods for producing spheroid cultures enriched with cancer stem cells, to kits and related products containing the cultures, and to uses of the cultures, including in drug screening.

### BACKGROUND TO THE INVENTION

The cancer stem cell (CSC) concept has important implications not only for our understanding of carcinogenesis, but also for the development of cancer therapeutics. There is a growing body of evidence showing that cancer stem cells contribute to chemotherapy and radiation resistance in cancer. The use of drugs that interfere with stem cell self-renewal represents the strategy of choice for novel effective anti-cancer treatments, but also a great challenge because cancer stem cells and their normal counterparts share many pathways.

The biology of cancer stem cells has proven complex and difficult to translate into effective therapeutic strategies. In order to monitor the effect of test compounds on cancer stem cells, a panel of markers, preferably easy-to-measure surface markers, must be defined. This is, however, cumbersome, since for many tumour indications that marker panel is not clearly defined, with often non-overlapping combinations of markers defining cell populations with cancer stem cell activities or tumour initiation ability. This is most likely reflecting the changing nature of the stemness capacity in tumour cells.

WO2015/158777 describes a method for preparing cells for 3D tissue culture. Cells are plated onto a suitable surface, cells which have not adhered to the surface are discarded, and the adherent cells are detached and transferred to a 3D tissue culture process.

The mammosphere assay was originally developed by Dontu et al. [1] as a way to select for and propagate mammary stem cells. Soon after the publication of this assay researchers started to use it as a reporter of stem cell and cancer stem cell activity from tissue samples, tumors and continuous cell lines [2], [3], [4]. There are a variant of human cellular models to study the molecular mechanisms of tumorigenesis and it has been shown that most of these cell lines contain a small subpopulation of cells displaying functional stem cell characteristics with tumorigenic capacity [4]. Moreover, it has been reported that mammosphere formation in breast carcinoma cell lines depends on expression of E-cadherin [5].

There remains an unmet need for cancer stem cell spheroids, particularly to supply cells in easy or read-to-use form for drug screening and other assays. Related to this there is a need for methods of preparing cultures rich in cancer stem cell spheroids or which have the propensity to form cancer stem cell spheroids. The present invention addresses these and other needs, as described in further detail herein.

### BRIEF DESCRIPTION OF THE INVENTION

Broadly, the present inventors have found that a cancer stem cell enriched cell population, formed from disaggregated 3D cultures, may be frozen as a single cell preparation in multi-well plates. The frozen cells are then readily thawed (e.g. after shipping) by adding warm cell culture medium and cells allowed to grow into spheroids. Without wishing to be bound by any particular theory, the present inventors believe that the disaggregated cancer stem cell enriched population retains “stemness” and therefore facilitates the preparation of 3D spheroids.

Accordingly, in a first aspect the present invention provides method for producing a population of ready-to-use spheroid forming cancer cells, comprising:
(i) growing cancer cells in suspension culture in a first culture medium on one or more first low-adhesion tissue culture plates thereby forming cancer cell spheroids enriched in cancer stem cells;
(ii) disaggregating said cancer cell spheroids to form a suspension of single cells enriched in cancer stem cells;
(iii) plating said suspension of single cells in a second culture medium on one or more second low-adhesion tissue culture plates; and
(iv) freezing said suspension of single cells in said one or more second tissue culture plates, thereby producing a population of ready-to-use spheroid forming cancer cells.

In some cases, said cancer cells comprise cells of a cancer cell line (e.g. an immortalised cell line) or primary cell culture derived from a tumour (e.g. a human or other animal tumour or a patient-derived xenograft). In particular, the cancer cell line may be selected from the group consisting of: human breast carcinoma MDA-MB-436; human glioblastoma U87MG; human colon carcinoma HCT116; human ovarian SK-OV-3; human lung NCI-H446; human lung A549 carcinoma; human pancreatic PANC1; human pancreatic Capan-1; human MCF-7 breast carcinoma; human BT474 breast carcinoma; human OVCAR ovarian carcinoma; human LNCaP Prostate carcinoma; and human CaCo2 colon carcinoma. Cell lines may be obtained commercially, e.g., from American Type Culture Collection (ATCC), 10801 University Boulevard, Manassas, Va. 20110, USA.

In some cases, said first culture medium comprises a mixture of Dulbecco's modified Eagle's medium (DMEM) and Ham's F-12 medium, supplemented with B27 supplement, epidermal growth factor (EGF) and basic fibroblast growth factor (bFGF). The first culture medium may be “Dontu medium” as defined in [1]. In some cases, the first culture medium comprises:
DMEM/F12 (1:2 mixture);
Methycellulose (final 5%);
B27 supplement (final 2%);
EGF (final 20 ng/ml); and
bFGF (final 20 ng/ml).

In some embodiments, the medium does not comprise methylcellulose.

In some embodiments in accordance with the first aspect of the invention, the EGF and bFGF supplementation is added only once at the beginning of the culture and is not added repeatedly thereafter. In some cases the EGF and bFGF supplementation is performed every 2 days.

In some cases, said one or more first low-adhesion tissue culture plates and/or said one or more second low-adhesion tissue culture plates comprise a coating of poly-2-hydroxyethylmethacrylate. In some cases, said one or more first low-adhesion tissue culture plates and/or said one or more second low-adhesion tissue culture plates comprise ultra-low attachment plates, such as Corning® Ultra-Low Attachment Spheroid Microplates (Corning Incorporated, Corning, N.Y., USA). Ultra-low attachment plates are available commercially and have a covalently bonded hydrogel surface that is non-cytotoxic and biologically inert.

In some cases, said one or more second low-adhesion tissue culture plates comprise more wells per plate than said one or more first low-adhesion tissue culture plates. For example, said one or more second low-adhesion tissue culture plates may comprise 96-well plates or 384-well plates; and/or said one or more first low-adhesion tissue culture plates comprise 6-well plates or 12-well plates. This way the first plates are suited to initial bulk phase production of spheroids, while the second plates are suited to carrying out assays (e.g. test compound screening) on spheroid formation and/or spheroid viability.

In some cases, said second culture medium comprises a cryopreservation medium. For example, CELLBANKER® cell freezing medium available commercially from AMS Biotechnology (Europe) Ltd, Milton Park, UK.

In some cases, growing said cancer cells in step (i) comprises plating the cells at a density of between 1000 cells/ml and 100000 cells/ml. In particular, as described in Example 2 herein, cell lines exhibit certain optimal plating densities for spheroid formation while minimizing spheroid aggregation. In particular embodiments, the cancer cells comprise breast carcinoma MDA-MB-436 cells and the growing said cancer cells in step (i) comprises plating the cells at a density of between 10000 cells/ml and 30000 cells/ml, optionally at a density of about 25000 cells/ml. In certain embodiments, the cancer cells comprise glioblastoma U87MG cells and the growing said cancer cells in step (i) comprises plating the cells at a density of between 5000 cells/ml and 15000 cells/ml, optionally at a density of about 10000 cells/ml.

In some cases in accordance with the first aspect of the present invention, said plating said suspension of single cells in step (iii) comprises plating the cells at a density of between 1000 cells/ml and 2000 cells/ml, optionally at a density of about 1600 cells/ml. In some cases in accordance with the first aspect of the present invention, for example when said cancer cells comprise PDX-derived cells, said plating said suspension of single cells in step (iii) comprises plating the cells at a density of between 5000 cells/well (standard 96-well plate well size) and 15000 cells/well (standard 96-well plate well size), optionally at a density of about 10000 cells/well (standard 96-well plate well size). It is specifically contemplated herein that a plating density, e.g. for plate formats bigger or smaller than 96-well format may be scaled accordingly in proportion to either the surface area of the well of the volume of the well. For example, plating density may be around 50000 cells/ml (based on a 96-well plate well volume of 200 μl), such as in the range 40000 to 60000 cells/ml. The appropriate number of cells per well can therefore be selected by the skilled person in accordance with the known dimensions of the plate and well selected for carrying out the method of the present invention.

In particular, as described in Example 3 herein, cell lines exhibit certain optimal plating densities for spheroid formation while minimizing spheroid aggregation. In some embodiments, the cancer cells comprise colon carcinoma HCT116 cells or glioblastoma U87MG cells and the plating density in step (iii) is about 1600 cells/ml. In particular, the PDX experiments described in Example 4 demonstrated that spheroid forming efficiency was superior at a plating density (step (iii)) of 10000 cells/well for T2 (passage 2) PDX-derived cells in comparison with 20000 cells/well. The experiments were performed in 96-well plates having a well volume of around 200 μl. This means that the plating density of 10000 cells/well equates to a density of approximately 50000 cells/ml. It is therefore contemplated herein that a plating density, e.g. for plate formats bigger or smaller than 96-well format may be scaled accordingly in proportion to either the surface area of the well of the volume of the well. For example, plating density may be around 50000 cells/ml, such as in the range 40000 to 60000 cells/ml. The appropriate number of cells per well can therefore be selected by the skilled person in accordance with the known dimensions of the plate and well selected for carrying out the method of the present invention.

In some cases, plating said suspension of single cells in step (iii) comprises plating the cells into multiple wells at different cell densities. In particular, the wells at the edge of the one or more second low-adhesion tissue culture plates may be left blank (i.e. without cells) or receive cells at a different density to wells not at the plate edge.

Also contemplated herein is the plating of multiple cell types (e.g. multiple cell lines) on a single plate. For example, a “breast cancer plate” may comprise two or more different breast cancer cell lines. Additionally or alternatively, a plate may comprise a range of cancers, e.g. breast, brain, lung, pancreas, colon and/or ovarian cell lines. Accordingly, the method of the first aspect of the invention may comprise plating in step (iii) at least a first suspension of single cells enriched in cancer stem cells of a first cancer cell type and a second suspension of single cells enriched in cancer stem cells of a second cancer cell type, wherein the first and second cancer cell types are plated into different wells.

Preferably plating of different cell types is done according to a predetermined pattern.

In some cases the method of the first aspect of the invention further comprises packing, labelling and/or shipping the one or more second tissue culture plates comprising the frozen population of ready-to-use spheroid forming cancer cells. Typically, the plates will be sealed and shipped in a container that maintains their frozen state.

In some cases, the method of the first aspect of the invention further comprises:
(v) thawing the frozen population of ready-to-use spheroid forming cancer cells by adding a third culture medium to some or all of the wells of the one or more second tissue culture plates and warming the one or more second tissue culture plates at least until the frozen cells are thawed. This may conveniently be achieved by adding warm cell culture medium to the plate and placing the plate into a cell culture incubator (e.g. at 37° C.).

In some cases, the third culture medium has the same composition as defined above in connection with the first culture medium.

In some cases, the method of the first aspect of the invention further comprises:
(vi) growing the thawed population of ready-to-use spheroid forming cancer cells until a plurality of spheroids form. This may in some cases comprise incubating the cells for between 5 and 7 days, optionally for about 6 days. As can be seen in Example 3 herein, day 6 of culture was found to be optimal in terms of spheroid number with minimal aggregation for the cell lines HCT116 and U87MG. Alternatively, in some cases, such as for example when the cancer cells comprise PDX-derived cells, the method may comprise incubating the cells for between 4 and 6 days, optionally for about 5 days. As can be seen in Example 4 herein, day 4 of culture was found to be superior in terms of spheroid number with minimal aggregation for the PDX-derived cells in comparison with day 9 of culture.

In some cases, the method of the first aspect of the invention further comprises adding at least one test compound to one or more of the wells prior to the formation of spheroids in order to assess the effect of the at least one test compound on spheroid formation. In particular, if spheroid formation is delayed and/or prevented by the test compound in comparison to that observed in the absence of the test compound, this indicates that the test compound potentially exhibits anti-cancer effects, particularly inhibition of cancer stem cell growth and/or tumour self-renewal and regrowth.

In some cases, the method of the first aspect of the invention further comprises adding at least one test compound to one or more of the wells once spheroids have formed in order to assess the effect of the at least one test compound on spheroid viability. In particular, if cancer spheroids are killed or reduced in size or number by the test compound in comparison to that observed in the absence of the test compound, this indicates that the test compound potentially exhibits anti-cancer effects, particularly the ability to penetrate and kill a 3D tumour.

In a second aspect the present invention provides a population of ready-to-use spheroid forming cancer cells produced or producible by a method according to the first aspect of the invention.

In some cases according to the second aspect of the invention the cells are frozen in one or more low-adhesion multi-well tissue culture plates. The one or more plates may be sealed and/or labelled for shipping.

In some cases, the cells are cell line cells selected from the group consisting of: human breast carcinoma MDA-MB-436; human glioblastoma U87MG; human colon carcinoma HCT116; human ovarian SK-OV-3; human lung NCI-H446; human lung A549 carcinoma; human pancreatic PANC1; human pancreatic Capan-1; human MCF-7 breast carcinoma; human BT474 breast carcinoma; human OVCAR ovarian carcinoma; human LNCaP prostate carcinoma; and human CaCo2 colon carcinoma.

In some cases, the cancer cells are PDX-derived cells. The population of ready-to-use spheroid forming cancer cells of the present invention, and method for producing such cells, has been tested and validated in PDX cancer cells from a variety of sources. In particular tumour models have been tested using colorectal, breast and pancreatic tumour sources. In certain cases, the PDX-derived cells, in accordance with any aspect of the present invention, may be colorectal, breast or pancreatic cancer cells (e.g. derived from a primary tumour of the type colorectal, breast or pancreatic.

In some cases, a single multi-well plate comprise two or more types of cancer cell. For example, the plate may comprise 2, 3, 4, 5, 6, 7, 8 or 9 of the above-listed human cancer cell lines and/or PDX cells.

In a third aspect the present invention provides a kit of parts, comprising:
one or more low-adhesion multi-well tissue culture plates having plated therein a frozen population of ready-to-use spheroid forming cancer cells produced or producible by a method according to the first aspect of the invention; and
a sealed container comprising cell culture medium that is suitable for growing cancer cell spheroids.

In some cases, the cell culture medium is as defined above in connection with the first cell culture medium of the first aspect of the present invention. In particular, the cell culture medium may comprise a mixture of Dulbecco's modified Eagle's medium (DMEM) and Ham's F-12 medium, supplemented with B27 supplement, epidermal growth factor (EGF) and basic fibroblast growth factor (bFGF).

The kit may additionally comprise an insert, label or manual with instructions for use of the cells in a spheroid formation assay or a 3D cell viability assay of one or more test compounds.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 shows the number of spheres per ml for different media formulations and supplement schedules on the breast carcinoma cell line MDA-MB-436.

FIG. 2 shows spheroid diameter (μm) for different media formulations and supplement schedules on the breast carcinoma cell line MDA-MB-436.

FIG. 3 shows spheroid circularity for different media formulations and supplement schedules on the breast carcinoma cell line MDA-MB-436.

FIG. 4 shows the number of spheres per ml for different media formulations and supplement schedules on the glioblastoma cell line U87MG.

FIG. 5 shows spheroid diameter (μm) for different media formulations and supplement schedules on the glioblastoma cell line U87MG.

FIG. 6 shows spheroid circularity for different media formulations and supplement schedules on the glioblastoma cell line U87MG.

FIG. 7 shows sphere number plotted against cell density (cells/ml) for the breast carcinoma cell line MDA-MB-436. As can be see, sphere number initially increases in a relatively linear fashion with cell density, but then reaches a plateau at around 25000 cells/ml plating density.

FIG. 8 shows representative micrographs of spheroids formed upon cell density seeding on the breast carcinoma cell line MDA-MB-436. The cell densities are as indicated (1000, 5000, 10000, 25000, 50000, 100000 and 250000 cells/ml).

FIG. 9 shows sphere number plotted against cell density (cells/ml) for the glioblastoma cell line U87MG. As can be see, sphere number initially increases at a steep slope with cell density, but then the gradient decreases beyond 10000 cells/ml plating density.

FIG. 10 shows representative micrographs of spheroids formed upon cell density seeding on the glioblastoma cell line U87MG. The cell densities are as indicated (2000, 5000, 10000, 25000, 50000 and 100000 cells/ml).

FIG. 11 shows spheroid number plotted against plating cell density in multi-well plates. As can be seen, the effect of cell seeding density on multi-well spheroid number production from spheroid enriched cultures on multi-well plates using the colon carcinoma cell line HCT116 allowed 1600 cells/ml to be selected as the optimum density.

FIG. 12 shows spheroid diameter (μm) plotted against plating cell density for spheroid enriched cultures on multi-well plates using the colon carcinoma cell line HCT116.

FIG. 13 shows spheroid number plotted against plating cell density in multi-well plates. As can be seen, the effect of cell seeding density on multi-well spheroid number production from spheroid enriched cultures on multi-well plates using the glioblastoma cell line U87MG allowed 1600 cells/ml to be selected as the optimum density. For this cell line, seeding density higher than 1600 cells/ml produced fewer spheres due to aggregation of existing spheres.

FIG. 14 shows spheroid diameter (μm) plotted against plating cell density for spheroid enriched cultures on multi-well plates using the glioblastoma cell line U87MG.

FIG. 15 shows spheroid number plotted against days in culture after seeding at a density of 1600 cells/ml on multi-well plates using the colon carcinoma cell line HCT116. As can be seen, the highest number of spheroids was observed on day 6.

FIG. 16 shows spheroid diameter (μm) plotted against days in culture after seeding at a density of 1600 cells/ml on multi-well plates using the colon carcinoma cell line HCT116.

FIG. 17 shows spheroid number plotted against days in culture after seeding at a density of 1600 cells/ml on multi-well plates using the glioblastoma cell line U87MG. Spheroid number increased up to day 8 and then decreased, indicating aggregation of spheroids. The optimum recording date was therefore selected as day 6.

FIG. 18 shows spheroid diameter (μm) plotted against days in culture after seeding at a density of 1600 cells/ml on multi-well plates using the glioblastoma cell line U87MG. Spheroid diameter increased exponentially indicating aggregation of spheroids beyond day 8.

FIG. 19 shows a schematic overview of the process workflow according to an embodiment of the present invention. A 2D monoculture is grown to bulk 3D culture spheroids, which are then disaggregated to form a single cell preparation in a culture media suitable for cell freezing. The cancer stem cell (CSC) enriched single cell population is then dispensed into 96-well plates, which are packaged and labelled appropriately before freezing at −80° C. The frozen ready to use spheroid-forming plate may then be shipped to an end user. The end user thaws the plate by adding warm cell culture medium and grows the cells for 5-7 days. The cells re-form spheroids.

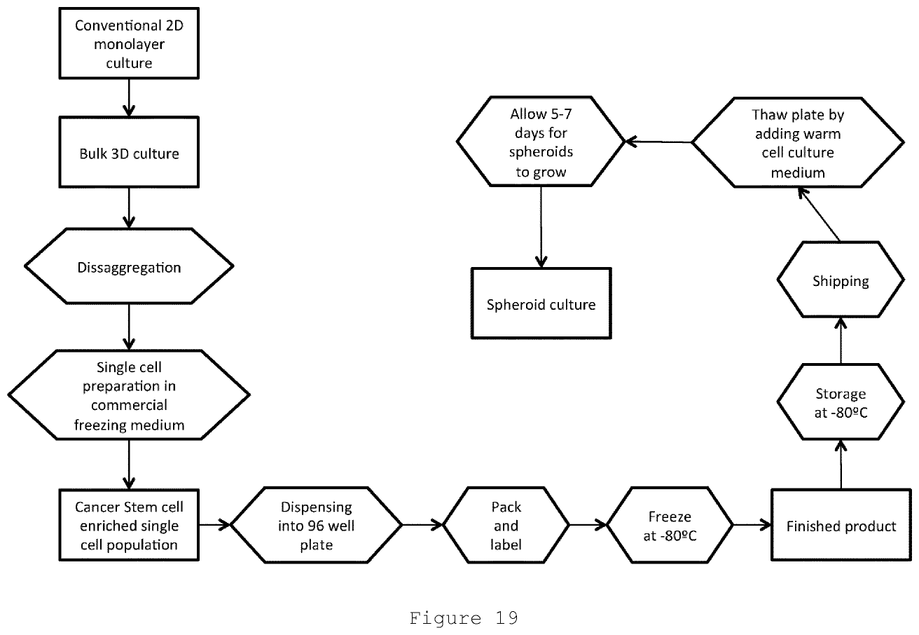

FIG. 20 shows a schematic overview of assay processes according to some embodiments of the present invention. The frozen ready to use spheroid forming plate is thawed by adding stem cell media. At this point test compounds can be added in order to assess their effect on spheroid formation (left-hand branch: “spheroid formation assay”). Additionally or alternatively, test compounds may be added after spheroids have formed in order to assess their effect on 3D spheroid viability (right-hand branch: “3D viability assay”).

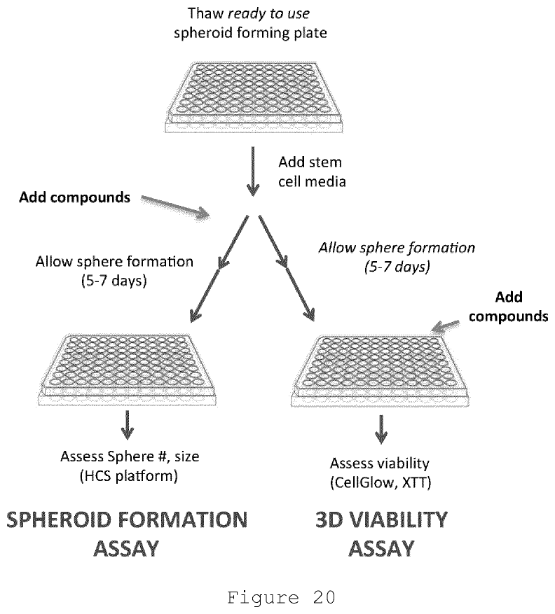

FIG. 21 shows a photomicrograph of representative spheres obtained from passage 1 (T1) PDX-derived cells shown at day 13. Diameter bars are 150 μm (lower left) and 182 μm (upper right).

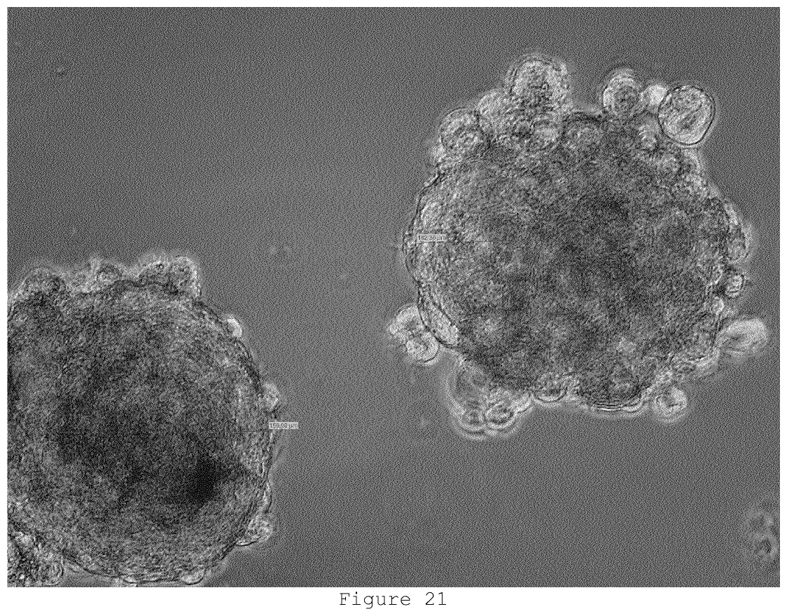

FIG. 22 shows representative whole well reconstruction images (2.5× magnification) from control (left) and thawed (right) Cell2Sphere™ plates at day 4 of spheroid growth at 10 000 cells/well.

### DETAILED DESCRIPTION OF THE INVENTION

In describing the present invention, the following terms will be employed, and are intended to be defined as indicated below.

“Cancer stem cell” (“CSC”) are tumorigenic cancer cells capable of self-renewal. Typically CSCs are able to grow in suspension culture and form cancer cell spheroids.

“Spheroid” as used herein refers to a spherical or sphere-like structure formed of cancer cells typically enriched with CSCs. Also called “tumorospheres” (see Weiswald et al., Neoplasia, 2015, Vol. 17, No. 1, pp. 1-15, the content of which is expressly incorporated herein by reference), the spheroid may be a free-floating structure cultured in low-adherent conditions using a serum-free medium supplemented with certain growth factors.

“Spheroid forming cells” as used herein refers to cancer cells, including CSCs, that exhibit the propensity to form spheroids upon culturing under appropriate conditions for an appropriate time. Therefore, the spheroid forming cells need not be in the form of a spheroid. Typically, the spheroid forming cells are derived from a spheroid, e.g., following enzymatic and/or mechanical dissociation to yield a suspension of single cells. The spheroid forming cells may be enriched in CSCs. Moreover, the spheroid forming cells may in some cases be frozen, e.g. in a cryopreservation medium.

“Test compound” as used herein refers to any agent having or which is a candidate for having an effect on a cell, tissue, organ or system of interest. In particular, the test compound may be any drug (whether small molecule, protein, biologic, nucleic acid or carbohydrate) that may, for example, be employed in screening for anti-cancer properties. Of particular, interest are test compounds that show efficacy against spheroid formation, spheroid growth and/or spheroid viability.

The following is presented by way of example and is not to be construed as a limitation to the scope of the claims.

### EXAMPLES

### Example 1—Effect of Cell Culture Medium Composition and Supplementation on Spheroid Formation

### Methodology

Standard medium (SM):
DMEM/F12 (1:2 mixture)
Methylcellulose (final 5%)
B27 supplement (final 2%)
EGF (final 20 ng/ml)
bFGF (final 20 ng/ml)

Standard supplementation procedure:
1. Mix base medium (DMEM+F12)
2. Add Methylcellulose
3. Add B27
4. Mix
5. Re-suspend cells in mix.

Add growth factors (EGF+bFGF) directly to the culture well every 2 days.

Incubation conditions: 37° C., 5% CO₂.

Disaggregation/dissociation of spheroids was enzymatic (5 min in 1:1 trypsin:DMEM solution at 37° C.) and mechanical (passing through a 25G needle (6 strokes).

As has been reported previously (e.g. [1] and [5]), certain human breast cancer cell lines are capable of forming spheroids (“mammospheres”). Cells are seeded in DMEM:F12 (2:1) medium without serum, supplemented with B27, EGF (20 ng/ml), bFGF (20 ng/ml) in six-well tissue culture plates that had been covered with poly-2-hydroxyethylmethacrylate (Sigma, St. Louis, Mo.) to prevent cell attachment, at a density of 1000 cells/ml. In order to prevent cell aggregation, methylcellulose was included in the medium at a final concentration of 0.5% (see [5]).

### Results

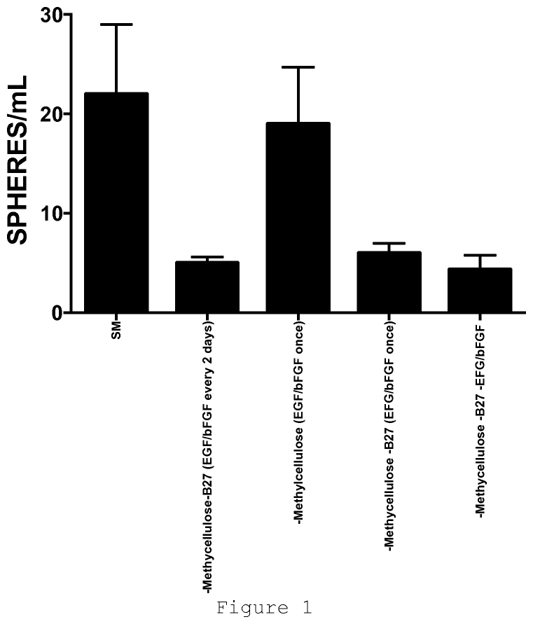

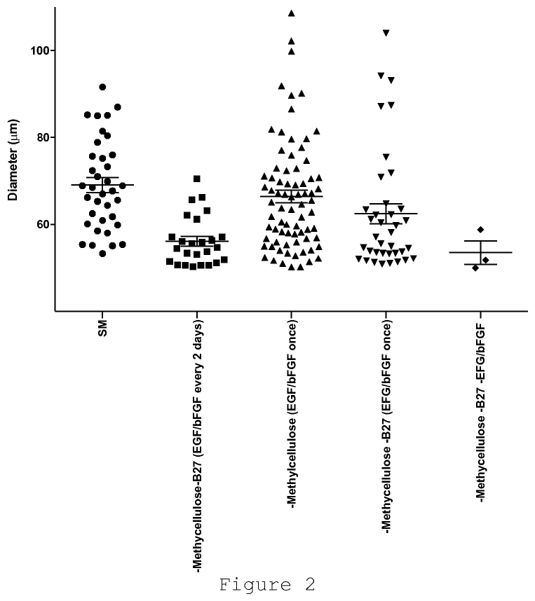

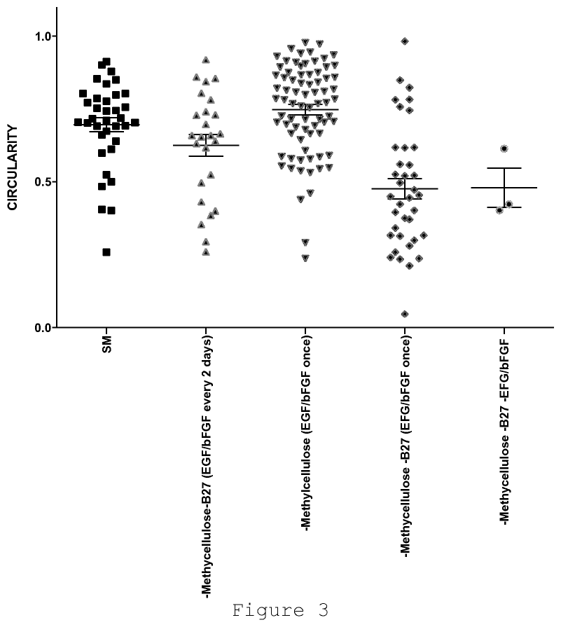

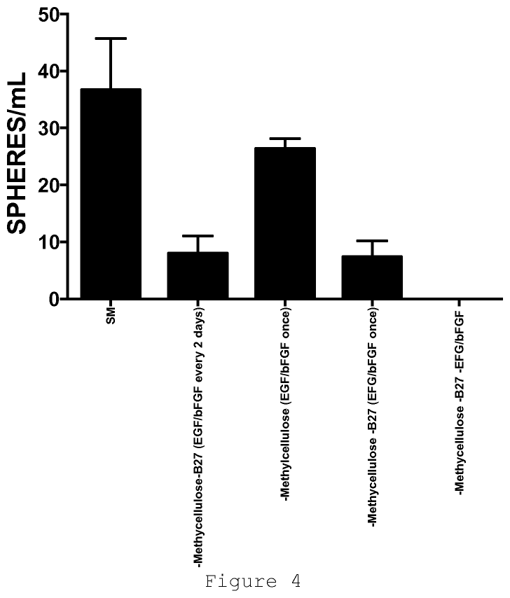

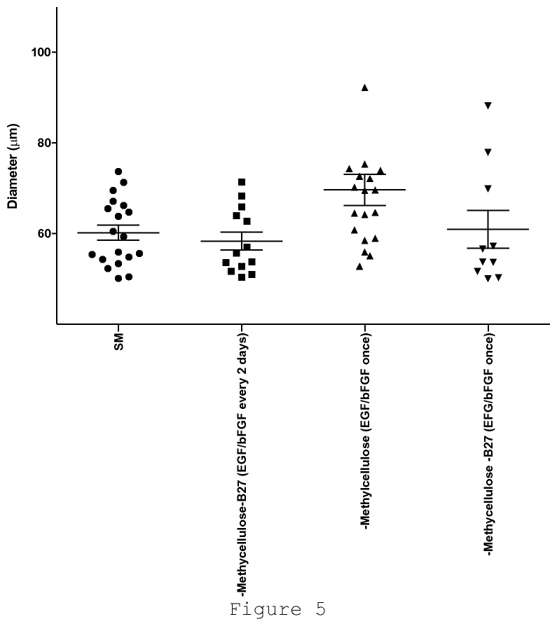

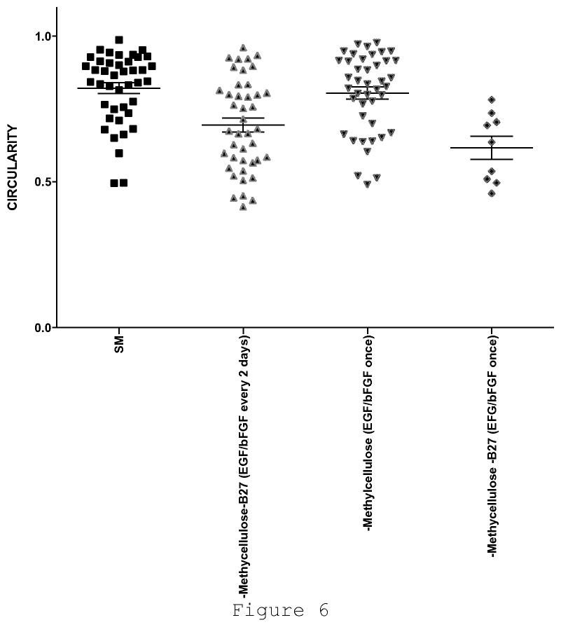

The effects of eliminating methylcellulose and changing growth factor (EGF and bFGF) supplementation to once at the beginning of the culture were assessed in relation to spheroid number, spheroid size and spheroid circularity using breast carcinoma and glioblastoma cell lines. As shown in FIGS. 1-6, it was found that methylcellulose can be eliminated from SM and growth factor supplementation carried out only once at the start of the culture while still maintaining a comparable number of spheroids (as compared with SM and standard supplementation procedure) and of approximately equal diameter and circularity (as compared with SM and standard supplementation procedure). By contrast, eliminating both methylcellulose and B27 led to lower numbers of spheroids for the cell lines investigated (MDA-MB-436 and U87MG).

### Example 2—Optimizing Conditions for Bulk-Phase (Phase 1) Spheroid Production

In order to enrich cell cultures for spheroid producing cells, spheroids are produced in bulk from single cells grown in monolayers (2D).

### Cell Density Optimization

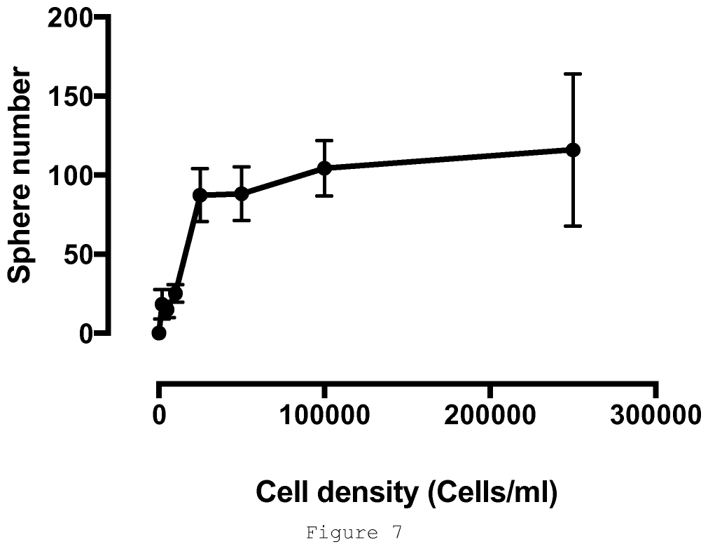

The number of cells/ml was tested against number of spheroids produced. We observed that upon a certain density spheroid number did not increased proportionally, indicating that spheroids aggregated. This can be seen from the supporting data on spheroid number and representative micrographs using breast carcinoma and glioblastoma cell lines presented in FIGS. 7-10. Therefore, cell density may optimally be maintained below this level, which is specific for each cell line.

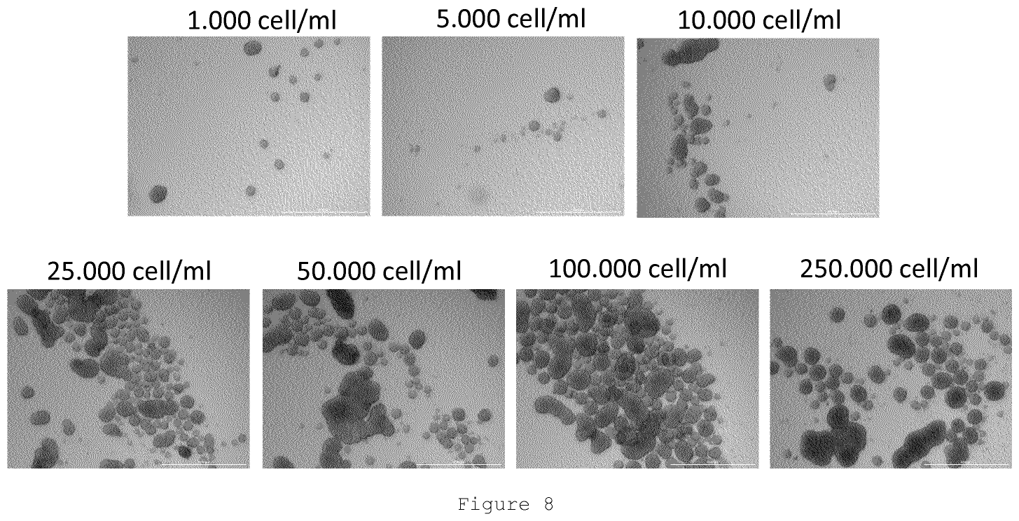

As shown in FIGS. 7 and 8, the seeding density of MDA-MB-436 cells may optimally be ≤25000 cells/ml.

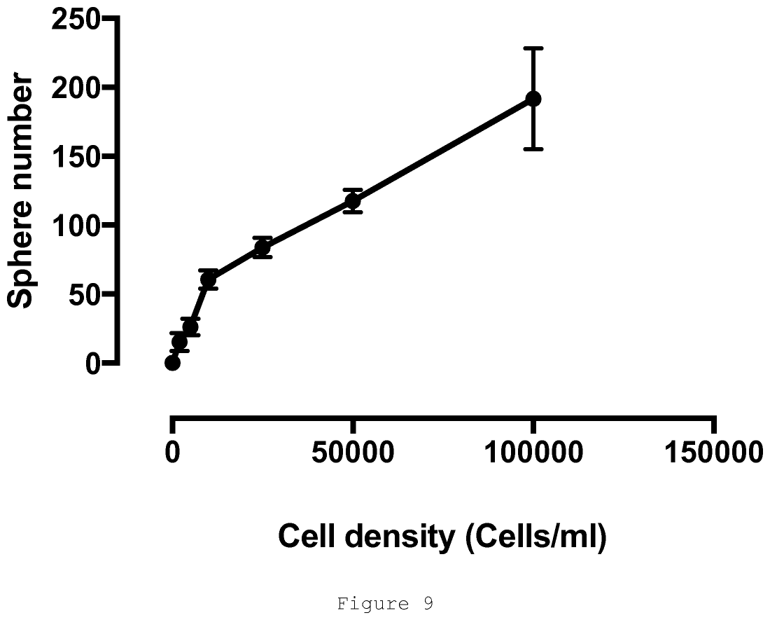

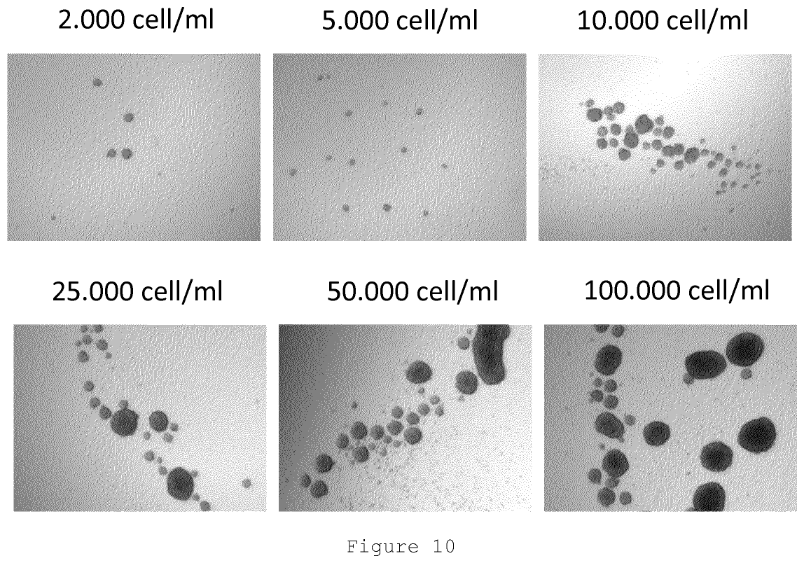

As shown in FIGS. 9 and 10, the seeding density of U87MG cells may optimally be ≤10000 cells/ml.

### Example 3—Optimizing Conditions for Spheroid Production Multi-Well Plates (Phase 2)

In accordance with certain embodiments of the present invention, spheroids prepared in phase 1 (as described above in Example 2) were mechanically and enzymatically disaggregated and filtered to render a single cell suspension. Cells were re-suspended in freezing medium (Cell Banker®, AMS Biotechnology (Europe) Ltd, Milton Park, UK) and dispensed 20 μl/well into 96-well plates. The plates may then be flash-frozen at −80° C. for storage. The cells are thawed by adding warm SM (or SM without methylcellulose) and grown in culture for 5-7 days to form spheroids.

The effect of plating cell density on spheroid production was assessed by plating cells at different densities and then measuring spheroid number and spheroid diameter after 7 days in culture.

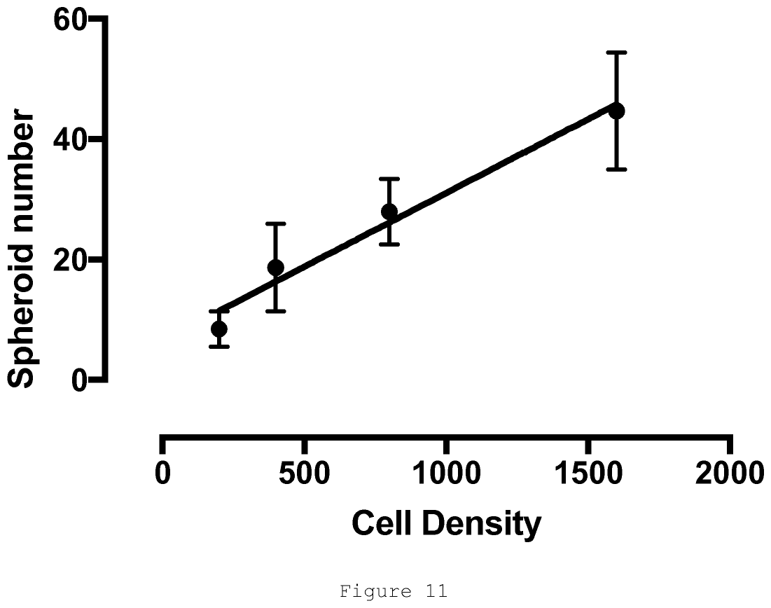

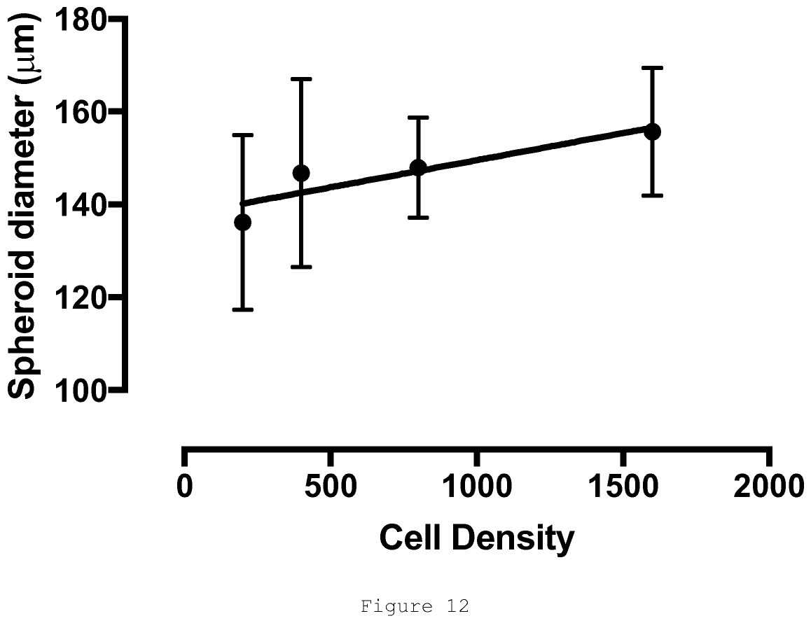

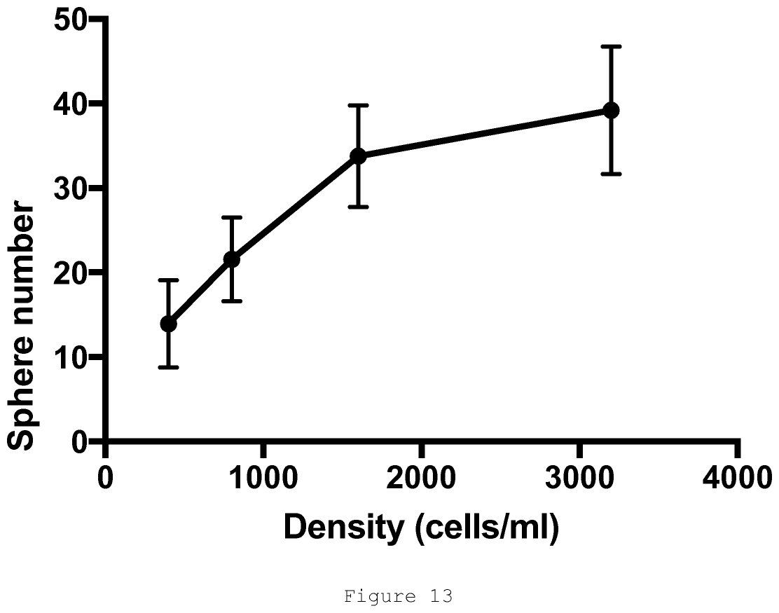

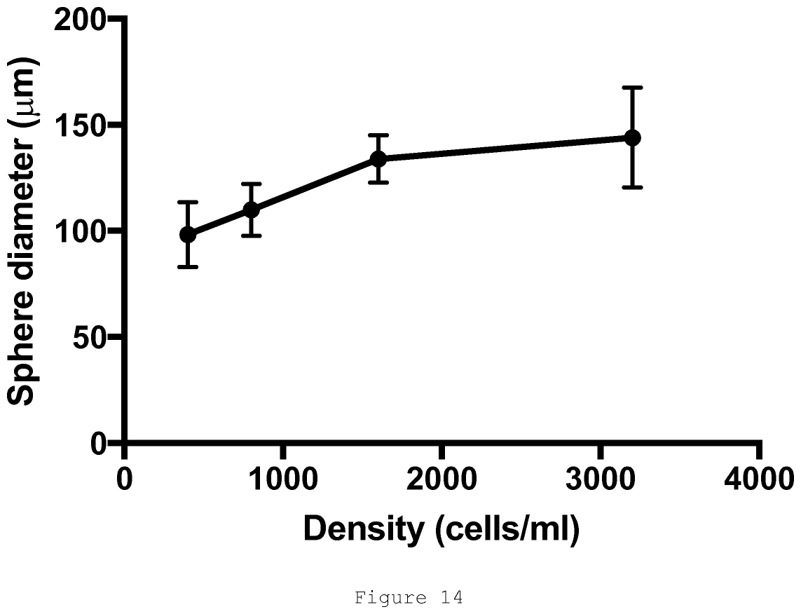

As can be seen from FIGS. 11-14, the number of spheroids produced increased in proportion to the plating cell density. This was initially linear, but at least for U87MG cells, this flattened off above 1600 cells/ml due to aggregation of existing spheroids. Therefore, it was concluded that the optimum plating cell density in this case is around 1600 cells/ml.

In order to test the optimum day for assay data recording, cell number and size were measured every day using a starting cell seeding density of 1600 cells/ml. Spheroid number stabilizes at a certain number of days, when all spheroid forming cells have grown to the spheroid state. Diameter continues to increase as spheroids grow.

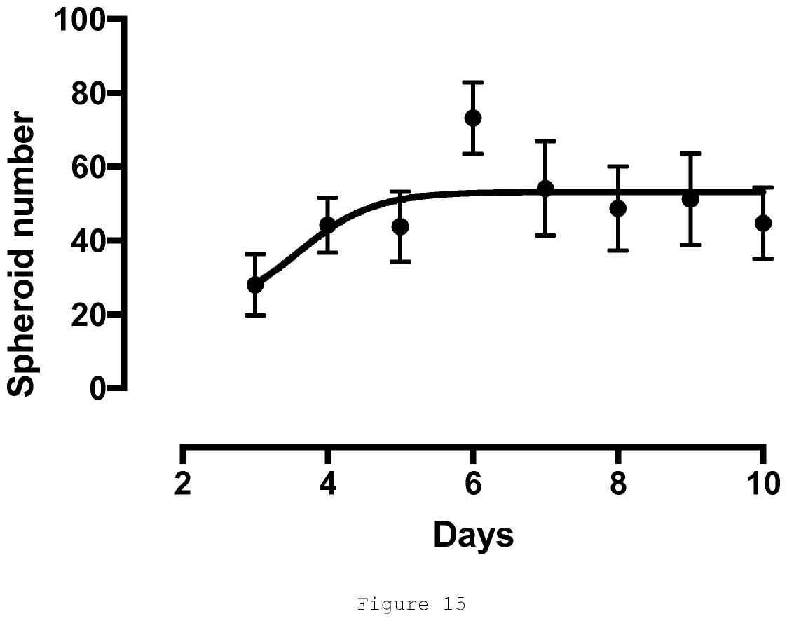

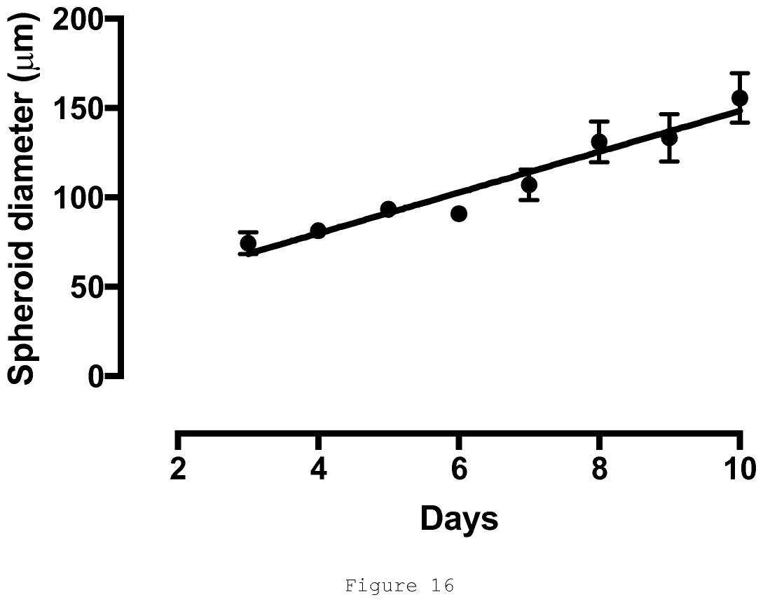

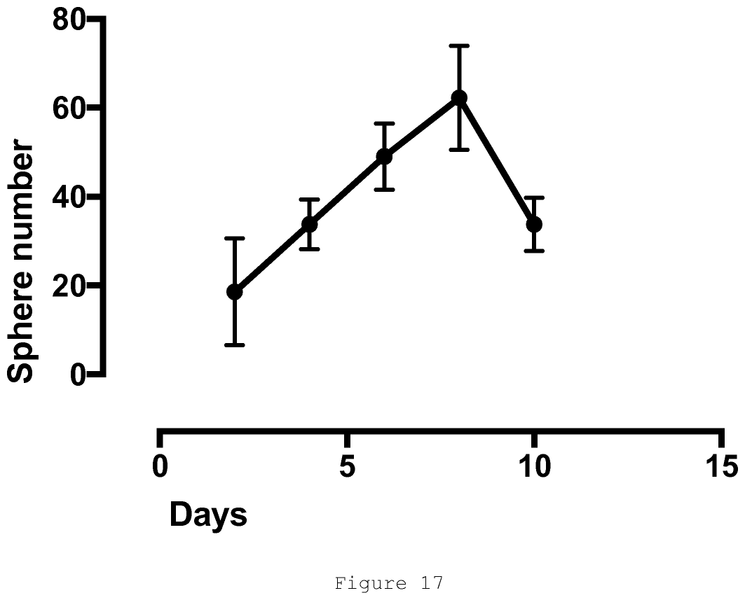

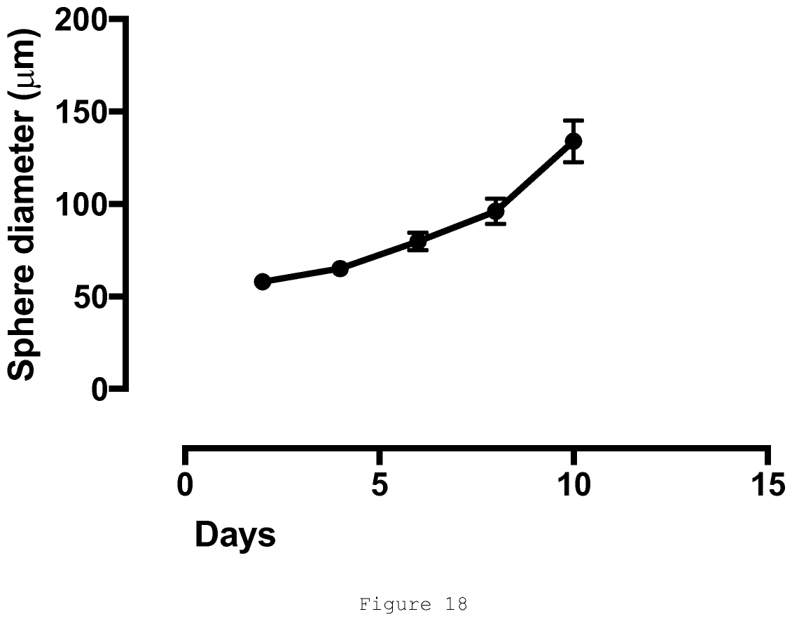

As shown in FIGS. 15-18, the spheroid number and diameter using colon carcinoma and glioblastoma cell lines was found to be optimal around day 6 of culture. Beyond this point spheroid aggregation became evident for HCT116 cells and U87MG cells.

### Example 4—Spheroid Production from Patient-Derived Xenograft (PDX) Cells in Multi-Well Plates

A sample colorectal tumor fragment was processed upon arrival into single cell suspension, obtaining a viability of 62.2% viable cells. 12×10⁶ spare cells were seeded in bulk plates at a cell density of 20000 cells/ml in Cell2Sphere™ medium for enrichment in CSC (T1 spheroids). This is the same medium as described in detail above in Example 1. After 13 days, spheroids were disaggregated enzymatically and mechanically to obtain single cell suspensions. The method of enzymatic and mechanical disaggregation (trypsin/DMEM+25G needle) was the same as described in detail above in Example 1. A total of 23532 spheroids were collected.

T1 spheroids were then disaggregated, obtaining 5.3×10⁶ viable cells that were then seeded on P96 plates in two different cell densities (10000 cells/well and 20000 cells/well) rendering 6 plates in total. 1 plate of each set was used as control, the rest were frozen at −80° C. for storage.

After 3 days, frozen plates were thawed, and then the plates were analyzed for sphere number and viability at day 4 and day 9, following Cell2Sphere™ instructions. The image analysis was as described for cell lines in Examples 1-3 above. Data obtained is summarized below (Table 1). These are named “T2” (for passage 2) spheroids.

TABLE 1
T2 spheroid characteristics

| | | Average Spheroid | | Average Spheroid | | Average Spheroid | |
|---|---|---|---|---|---|---|---|
| | | No (per well) | sd | Size (μm) | sd | Circularity | sd |
| 10,000 cell/well | Control | 35.92 | 13.15 | 93.71 | 11.66 | 0.71 | 0.03 |
| | Frozen, Day 4 | 70.20 | 19.71 | 78.43 | 8.26 | 0.70 | 0.02 |
| | Frozen, Day 9 | 30.53 | 8.24 | 100.08 | 12.67 | 0.70 | 0.02 |
| 20,000 cell/well | Control | 59.40 | 10.84 | 100.49 | 8.23 | 0.72 | 0.02 |
| | Frozen, Day 4 | 82.09 | 22.22 | 87.31 | 13.56 | 0.71 | 0.02 |
| | Frozen, Day 9 | 27.27 | 12.18 | 125.20 | 26.31 | 0.71 | 0.03 |

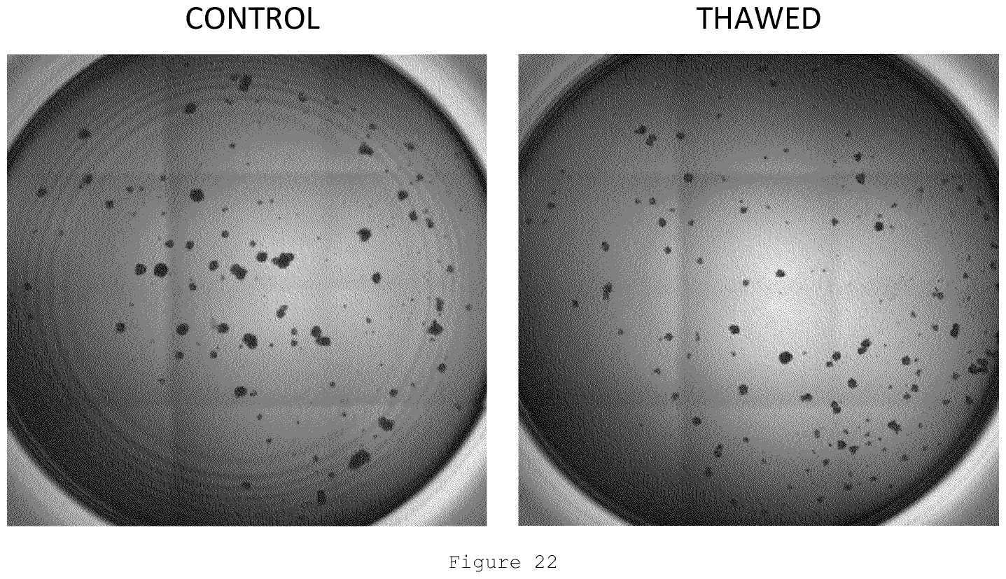

Representative images of wells from the P96 plates are shown in FIG. 22.

Spheroid aggregates were observed when spheroids were grown for 9 days in Cell2Sphere™ plates, which explains the decrease in spheroid number from day 4 to day 9 (see Table 1 above). Without wishing to be bound by any particular theory, the present inventors believe that 4-6 days is optimum for PDX-derived spheroid number scoring.

Spheroid forming efficiency was calculated as 1/510 for T1 spheroids and 1/143 for T2 at 10000 cells/well and 1/244 at 20000 cells/well. Therefore, under the conditions tested the best enrichment was obtained at 10000 cells/well in T2 using Cell2Sphere™ technology.

The standard deviation seen in the present experiments was relatively high (e.g. 19.7% for spheroid number in frozen day 4 10000 cells/well—see Table 1). However, it is expected that variability of cell number, and therefore standard deviation will decrease as industrial production processes are applied.

Using these conditions as estimation, it is possible to generate 8 plates from 12×10⁶ single viable cells from fresh samples. Given that the average sample renders 20×10⁶ single viable cells/gram of tumor, it will be possible to generate 13-14 Cell2Sphere™ plates per gram of fresh tumor tissue. Some variation is, however, expected from sample to sample.

### CONCLUSIONS

Given the results described above, we conclude that fresh PDX samples can be used to produce frozen viable Cell2Sphere™ plates for spheroid generation for drug testing ex vivo.

### REFERENCES

[1] Dontu G, Abdallah W M, Foley J M, et al. In vitro propagation and transcriptional profiling of human mammary stem/progenitor cells. Genes Dev 2003; 17:1253-70.
[2] Ponti D, Costa A, Zaffaroni N, et al. Isolation and in vitro propagation of tumorigenic breast cancer cells with stem/progenitor cell properties. Cancer Res 2005; 65:5506-11.
[3] Grimshaw M J, Cooper L, Papazisis K, et al. Mammosphere culture of metastatic breast cancer cells enriches for tumorigenic breast cancer cells. Breast Cancer Res 2008; 10:R52.
[4] Fillmore C M, Kuperwasser C. Human breast cancer cell lines contain stem-like cells that self-renew, give rise to phenotypically diverse progeny and survive chemotherapy. Breast Cancer Res 2008; 10:R25.
[5] Manuel Iglesias J, et al. Mammosphere formation in breast carcinoma cell lines depends upon expression of E-cadherein. PLoS ONE, 2013; 8(10); e77281.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

1. A method for producing a population of ready-to-use spheroid forming cancer cells, comprising:
(i) growing cancer cells in suspension culture in a first culture medium on one or more first low-adhesion tissue culture plates thereby forming cancer cell spheroids enriched in cancer stem cells;
(ii) disaggregating said cancer cell spheroids to form a suspension of single cells enriched in cancer stem cells;
(iii) plating said suspension of single cells in a second culture medium on one or more second low-adhesion tissue culture plates; and
(iv) freezing said suspension of single cells in said one or more second tissue culture plates, thereby producing a population of ready-to-use spheroid forming cancer cells.

2. The method according to claim 1, wherein said cancer cells comprise cells of a cancer cell line or primary cell culture derived from a tumour.

3. The method according to claim 2, wherein the cancer cell line is selected from the group consisting of: human breast carcinoma MDA-MB-436; human glioblastoma U87MG; human colon carcinoma HCT116; human ovarian SK-OV-3; human lung NCI-H446; human lung A549 carcinoma; human pancreatic PANC1; human pancreatic Capan-1; human MCF-7 breast carcinoma; human BT474 breast carcinoma; human OVCAR ovarian carcinoma; human LNCaP prostate carcinoma; and human CaCo2 colon carcinoma.

4. The method according to claim 2, wherein said primary culture comprises a patient derived xenograft (PDX).

5. The method according to any one of the preceding claims, wherein said first culture medium comprises a mixture of Dulbecco's modified Eagle's medium (DMEM) and Ham's F-12 medium, supplemented with B27 supplement, epidermal growth factor (EGF) and basic fibroblast growth factor (bFGF).

6. The method according to claim 5, wherein the medium does not comprise methylcellulose.

7. The method according to claim 5 or claim 6, wherein the EGF and bFGF supplementation is added only once at the beginning of the culture and is not added repeatedly thereafter.

8. The method according to any one of the preceding claims, wherein said one or more first low-adhesion tissue culture plates and/or said one or more second low-adhesion tissue culture plates comprise a coating of poly-2-hydroxyethylmethacrylate or comprise Corning® Ultra-Low Attachment plates.

9. The method according to any one of the preceding claims, wherein said one or more second low-adhesion tissue culture plates comprise more wells per plate than said one or more first low-adhesion tissue culture plates.

10. The method according to any one of the preceding claims, wherein:
said one or more second low-adhesion tissue culture plates comprise 96-well plates or 384-well plates; and/or
said one or more first low-adhesion tissue culture plates comprise 6-well plates or 12-well plates.

11. The method according to any one of the preceding claims, wherein said second culture medium comprises a cryopreservation medium.

12. The method according to claim 11, wherein said cryopreservation medium comprises CELLBANKER® cell freezing medium.

13. The method according to any one of the preceding claims, wherein growing said cancer cells in step (i) comprises plating the cells at a density of between 1000 cells/ml and 100000 cells/ml.

14. The method according to claim 13, wherein the cancer cells comprise breast carcinoma MDA-MB-436 cells and the growing said cancer cells in step (i) comprises plating the cells at a density of between 10000 cells/ml and 30000 cells/ml, optionally at a density of about 25000 cells/ml.

15. The method according to claim 13, wherein the cancer cells comprise glioblastoma U87MG cells and the growing said cancer cells in step (i) comprises plating the cells at a density of between 5000 cells/ml and 15000 cells/ml, optionally at a density of about 10000 cells/ml.

16. The method according to any one of the preceding claims, wherein said plating said suspension of single cells in step (iii) comprises plating the cells at a density of between 1000 cells/ml and 2000 cells/ml, optionally at a density of about 1600 cells/ml.

17. The method according to any one of claim 16, wherein the cancer cells comprise colon carcinoma HCT116 cells or glioblastoma U87MG cells.

18. The method according to any one of claims 4 to 12, wherein the cancer cells comprise PDX cells and the growing said cancer cells in step (iii) comprises plating the cells at a density of between 5000 cells/well and 15000 cells/well, optionally at a density of about 10000 cells/well.

19. The method according to any one of the preceding claims, wherein said plating said suspension of single cells in step (iii) comprises plating the cells into multiple wells at different cell densities.

20. The method according to claim 19, wherein wells at the edge of the one or more second low-adhesion tissue culture plates are left blank or receive cells at a different density to wells not at the plated edge.

21. The method according to any one of the preceding claims, further comprising packing, labelling and/or shipping the one or more second tissue culture plates comprising the frozen population of ready-to-use spheroid forming cancer cells.

22. The method according to any one of the preceding claims, further comprising:
(v) thawing the frozen population of ready-to-use spheroid forming cancer cells by adding a third culture medium to some or all of the wells of the one or more second tissue culture plates and warming the one or more second tissue culture plates at least until the frozen cells are thawed.

23. The method according to claim 22, wherein the third culture medium is the same as the first culture medium.

24. The method according to claim 22 or claim 23, further comprising:
(vi) growing the thawed population of ready-to-use spheroid forming cancer cells until a plurality of spheroids form.

25. The method according to claim 24, wherein step (vi) comprises incubating the cells for between 5 and 7 days, optionally for about 6 days.

26. The method according to claim 24, wherein the cancer cells comprise PDX cells and wherein step (vi) comprises incubating the cells for between 4 and 6 days.

27. The method according to any one of claims 22 to 26, comprising adding at least one test compound to one or more of the wells prior to the formation of spheroids in order to assess the effect of the at least one test compound on spheroid formation.

28. The method according to any one of claims 24 to 27, comprising adding at least one test compound to one or more of the wells once spheroids have formed in order to assess the effect of the at least one test compound on spheroid viability.

29. A population of ready-to-use spheroid forming cancer cells produced or producible by a method as defined in any one of claims 1 to 21.

30. The population of cells according to claim 29, wherein the cells are frozen in one or more low-adhesion multi-well tissue culture plates.

31. The population of cells according to claim 30, wherein the one or more plates are sealed and/or labelled for shipping.

32. The population of cells according to any one of claims 29 to 31, wherein the cells are cell line cells selected from the group consisting of: human breast carcinoma MDA-MB-436; human glioblastoma U87MG; human colon carcinoma HCT116; human ovarian SK-OV-3; human lung NCI-H446; human lung A549 carcinoma; human pancreatic PANC1; human pancreatic Capan-1; human MCF-7 breast carcinoma; human BT474 breast carcinoma; human OVCAR ovarian carcinoma; human LNCaP prostate carcinoma; and human CaCo2 colon carcinoma.

33. The population of cells according to any one of claims 29 to 31, wherein the cancer cells are PDX cells.

34. A kit of parts, comprising:
one or more low-adhesion multi-well tissue culture plates having plated therein a frozen population of ready-to-use spheroid forming cancer cells produced or producible by a method as defined in any one of claims 1 to 21; and
a sealed container comprising cell culture medium that is suitable for growing cancer cell spheroids.

35. The kit according to claim 34, wherein the cell culture medium comprises a mixture of Dulbecco's modified Eagle's medium (DMEM) and Ham's F-12 medium, supplemented with B27 supplement, epidermal growth factor (EGF) and basic fibroblast growth factor (bFGF).
